(19) United States
(12) Patent Application Publication
(10) Pub. No.: US 2004/0186032 A1
(43) Pub. Date: Sep. 23, 2004

(54) Translucent soap bars and process for making same

(75) Inventors: Miguel A. Leone, Vernon, NJ (US); Ismail I. Walele, Saddle Brook, NJ (US)

Correspondence Address: WEINGRAM & ASSOCIATES P.C., P.O. BOX 927, MAYWOOD, NJ, 07607, US

(73) Assignee: FINETEX, INC.

(21) Appl. No.: 10/801,174

(22) Filed: Mar. 15, 2004

Related U.S. Application Data
(60) Provisional application No. 60/455,384, filed on Mar. 17, 2003.
(60) Provisional application No. 60/477,690, filed on Jun. 11, 2003.

(51) Int. Cl. A61K 7/50

(52) U.S. Cl. 510/147

(57) ABSTRACT

Translucent soap compositions comprising soap base and a translucency-enhancing amount of benzoate ester are disclosed. The benzoate ester has the general structure:

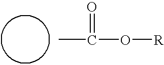

where R is a C12-C15 Alkyl, an alkyl group of C8 to C20, or a phenylethyl group. The translucent soap composition may further comprise a surfactant, namely, nonoxynol-10 carboxylic acid. Also disclosed is a method of making translucent soap compositions and a method of improving the translucency of opaque, translucent and transparent soap base.

### BACKGROUND OF THE INVENTION

1. Field of the Invention

This invention relates generally to translucent soap bars and methods for preparing same by batch or continuous processes. More particularly, this invention relates to making extruded or stamped translucent soap bars for bathing, hand, and face washing.

2. Description of the Related Art

Three main types of soap bars are known: opaque (non-transparent) soap; translucent soap; and transparent soap, which has ultimate translucency. Transparent and translucent soaps are becoming an important and steadily increasing segment of the soap market. Consumers perceive clear or translucent soap as somehow more pure and natural than opaque soap, believing it contains less undesirable components and is more easily rinsed from the skin after washing. However, transparent soap bars are expensive to manufacture.

The difference between non-transparent soaps and transparent or translucent soaps is the crystallization of the finished soap. Non-transparent soaps solidify to a crystal conglomerate, while crystallization is inhibited to a greater or lesser degree in translucent soap. Translucent soap bars are not as clear as transparent bars. A soap bar is considered translucent when it has a 40% to 85% opacity, and is considered opaque when it has over 85% opacity.

Various types of processes are used in making translucent soap. Casting or pouring by batch processing are the most traditional methods of making translucent soap, but are not widely used. Over the last decades, manufacturing of translucent soaps by extrusion has become possible. This method is gaining popularity because of the relative simplicity of the process, reducing the cost of manufacturing to an acceptable level. This lower cost of the product permits its availability to a larger group of consumers who are very much interested in this kind of soap because of its aesthetically very attractive form.

Translucent soaps have been formulated using several well-known methods, either by adding transparency-enhancing additives or agents to inhibit soap crystallization. For example, clarifying agents such as lower alkanols, glycerin and/or sugar were added to inhibit soap crystallization, and the soaps were framed, not milled and plodded. Other methods include low temperature saponification of fats and oils pre-dissolved in warm alcohol, water, and glycerine, followed by evaporation of part of the alcohol/water azeotrope. Yet another method involves the addition of a polyhydric alcohol, such as glycerol, glycol, sugar or the like to a “neat soap” or semi-boiled soap, or to soap prepared by the cold process technology. Numerous other transparent and translucent soap bars are also known in the art which are prepared using additives or specialized equipment.

U.S. Pat. Nos. 4,791,097 and 5,959,130, both assigned to Finetex, Inc. of Elmwood Park, N.J., the assignee herein, all describe benzoic acid esters useful as emollients, solubilizers, solvents, plasticizers, and the like, for personal care compositions. Translucent combination soap-synthetic detergent soap bars and their methods of manufacture are disclosed in assignee's U.S. Pat. No. 4,963,284 to Novakovic et al. The entire disclosure of all these patents are incorporated herein by reference.

Although the prior art teaches preparation of translucent and transparent soap bars, the processes and products are often unsatisfactory. Crystallization inhibitors sometimes made the soap malodorous, resulted in the development of hard specks in the soap, or negatively affected other properties of the soap, such as its feel, or caused the soap to be too mushy when wet. Some additives had a tendency to evaporate during processing and storage, causing the bar soap to lose its translucency. Costly, specialized equipment was necessary to break down the crystal size of the finished soap bar. None of the prior art references or methods teach or suggest the specific translucent soap bars of this invention or its method of manufacture.

It has been found, completely surprisingly, that certain benzoate esters or combinations of two or more of these esters, when properly incorporated in a suitable soap base, act as crystallization inhibitors and result in the production of translucent soap bars, using known processes for making commercially available milled and plodded soaps. It is possible to prepare a translucent soap bar without addition of traditional crystalline inhibitors or use of specialized, costly equipment, in an extrudable manner, by combining a suitable, regular opaque soap base with suitable benzoate esters, by shear mixing, milling and plodding, without special treatment steps being necessary after mixing of the constituents, to obtain and maintain a translucent soap bar for personal care applications.

The preferred benzoate esters are sold by Finetex, Inc. of Elmwood Park, N.J. under the trade names:

a) FINSOLV® TPP (INCI name: C12-15 alkyl benzoate (and) dipropylene glycol dibenzoate (and) PPG-15 stearyl ether benzoate) (U.S. Pat. No. 4,791,097);

b) FINSOLV® TN (INCI name: C12-15 alkyl benzoate);

c) FINSOLV® BCO-115 (INCI name: castor oil benzoate)(U.S. Pat. No. 5,959,130);

d) FINSOLV® G-2 (INCI name: C16-17 alkyl benzoate); or

e) FINSOLV® BOD (INCI name: Octyl Dodecyl benzoate);

f) FINSOLV® SUN (INCI name: Phenylethyl benzoate); and

g) a combination of the above-identified benzoate esters,

in percentages ranging from 2% to 8%.

It has also been found that benzoate ester(s) in combination with a surfactant, namely Nonoxynol-10 Carboxylic Acid, sold under the trade name Surfine AZI-A by Finetex, Inc., is effective in enhancing the translucency of opaque, translucent, and transparent soap bases. Specifically, it has been found that a combination of Nonoxynol-10 Carboxylic Acid and at least one of FINSOLV® TPP (INCI name: C12-15 alkyl benzoate (and) dipropylene glycol dibenzoate (and) PPG-15 stearyl ether benzoate) or FINSOLV® SUN (INCI name: Phenylethyl benzoate) improves the translucency of soap compositions comprising soap base.

Furthermore, certain benzoate esters or combinations thereof, such as the above-described esters sold by Finetex, Inc., when properly incorporated in the saponification or neutralization process to produce a soap base, are believed to act as crystallization inhibitors and result in the production of translucent soap bars, using known processes for making commercially available milled and plodded soaps. The benzoate esters may be added at various points in the saponification or neutralization process, depending on the particular process and equipment used, to optimize translucency.

Certain benzoate esters or combinations thereof, including those set forth above, are believed useful as clarifying agents to improve the translucency of opaque, translucent and/or transparent soap bases and soap bars, so as to markedly improve their translucency.

Finsolv® TPP, a benzoate ester emollient/lubricant, is a mixture of three benzoate esters, that have the following structure:

A. Finsolv® TN, C12-15 Alkyl Benzoate, CAS #: 68411-27-4, whose structure is:

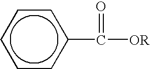

(where R is a C12-15 Alkyl)

B. Finsolv® PG-22, Dipropylene Glycol Dibenzoate, CAS #: 27138-31-4, whose structure is:

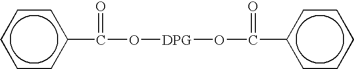

and

C. Finsolv® P PPG-15 Stearyl Ether Benzoate, CAS #: 108347-90-6, whose structure is:

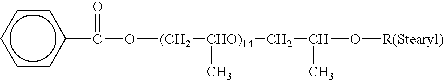

Finsolv® BCO-115 is a benzoate ester having the following structure:

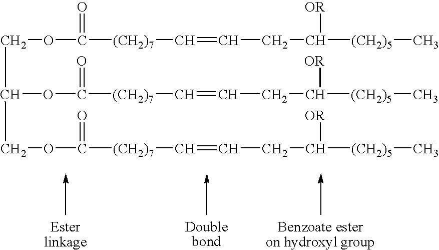

where: R=C₆H₅CO

FINSOLV® G-2 is the C16-C17 alkyl homologue of Finsolv® TN (C12-15 Alkyl Benzoate) and comprises a C16-C17 alkyl benzoate ester of branched monomethyl C16 and C17 alcohol which has the following structure:

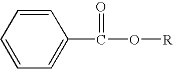

where R=monomethyl branched C16-C17 alkyl.

FINSOLV® BOD (INCI name: Octyl Dodecyl benzoate) is a benzoate ester having the following structure:

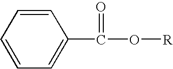

(where R is a C20 branched alkyl)

FINSOLV® SUN (INCI name: Phenylethyl benzoate) is a benzoate ester having the following structure:

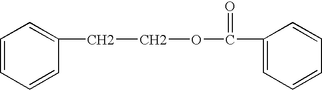

Broadly speaking, benzoate esters having the following general structure:

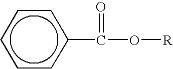

where R is a C12-C15 Alkyl, an alkyl group of C8 to C20, or a phenylethyl group

are believed to be useful compounds to improve translucency of opaque, translucent and transparent soap bases and soap bars. These compounds are also suitable as clarifying agents to clarify a clear base to improve translucency even more, and to clarify opaque or translucent soap bars or soap bases so as to significantly improve their translucency.

Surfine AZI-A (INCI name: Nonoxynol-10 Carboxylic Acid) is a surfactant which, added to soap base in combination with benzoate esters selected from the group consisting of Finsolv TPP and Finsolv SUN, has been found to improve translucency of the resulting soap composition. The structure of Surfine AZI-A (INCI name: Nonoxynol-10 Carboxylic Acid) is:

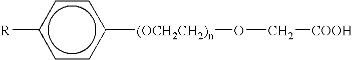

where R=C9 alkyl

n=10 moles Ethylene oxide

There are many advantages to the process of the invention, the most important being cost.

The present cost of translucent soap base is $0.70 per lb. as compared to $0.29 per lb. for regular opaque soap base. Using the process of the invention, it is possible to prepare translucent soap bars using opaque soap base costing $0.29/lb. It is estimated that the cost of producing a translucent soap bar according to the process of the invention ranges between $0.35 per lb. to $0.45 per lb., which is about half the cost of producing commercially available translucent soap bars. Thus, using the process of the invention, the cost of producing a translucent soap bar has been reduced by approximately 50%.

Another advantage of the process of the present invention is that the processing parameters are not as critical as those of known processes for producing translucent soap bars. Using the process of the invention, it is possible to produce translucent soap bars using conventional soap finishing equipment, without modifications to the standard equipment, and only slight adjustment to operating parameters in terms of optimal temperature and conditions.

Addition of these benzoate esters have other beneficial effects besides improving translucency, such as improving emolliency and skin softening properties and improving lathering characteristics of the soap.

### OBJECTS AND SUMMARY OF THE INVENTION

It is an object of this invention to provide a process for preparing hard, translucent soap bars which are significantly less expensive than similar bars prepared by known processes.

It is a further object of this invention to provide a process for the manufacture of translucent soap bars without the addition of costly additives or the use of specialized equipment to reduce crystallization.

It is an object of this invention to provide a milled or machined, or extruded or stamped soap bar which is translucent.

It is a further object of this invention to provide methods for preparing soap bars having enhanced clarity and translucency using conventional soap processing equipment.

Yet another object of the invention is to provide a high speed, continuous process for producing translucent soap bars in which the process parameters are not as critical as those of known processes.

It is still another object of the invention to provide translucent soap bars having superior properties, namely, emolliency, superior lathering and foam characteristics, and improved skin feel as compared to other soap bars.

Yet another object of the invention is to provide a method for preparing soap bars having improved translucency by the addition of certain benzoate esters at optimum points during the saponification or neutralization process to produce soap.

A still further object of the invention is to improve the translucency of opaque, translucent and/or transparent soap bars by the use of certain benzoate esters as clarifying agents.

Other objects and advantages will be apparent from the following detailed description of the invention.

These and other objects are obtained in one embodiment of the invention by combining a regular, opaque soap base with specified benzoate esters. The translucent soap bars so produced include many unique effects as compared to commercially available soap bars. The invention is further directed to a method of enhancing the translucency of an opaque soap base comprising incorporating in said soap base a translucency-enhancing amount of specified benzoate esters. The invention is also directed to continuous methods of producing said translucent soap bars.

In a second embodiment of the invention, these objects are obtained by incorporation of a translucency enhancing amount of specified benzoate esters in the saponification or neutralization process to produce a soap base. In a third embodiment of the invention, these objects are achieved by the use of a translucency-enhancing amount of specified benzoate esters as clarifying agents to clarify clear soap bars, so as to markedly improve their translucency.

### DETAILED DESCRIPTION OF THE INVENTION

In a first embodiment of the invention, the translucent soap bars of the invention may be made from regular opaque soap bases. The precise composition of the regular soap base used to prepare the translucent soap bars of the invention is not critical, and may comprise any regular soap base typically used in the manufacture of bar soaps, so long as the composition is not found to impair or inhibit translucency of the final soap bar products. Broadly stated, any regular soap base could be used as the starting ingredient for the process of the invention, in any percentage or combination as is known in the art. By “regular” soap base is meant opaque tallow/coconut oil soap bases and vegetable opaque soap bases. To produce translucent soap bars, applicants have found that the opaque soap base used as the starting ingredient in the process of the invention must meet two conditions. That is, the opaque soap base must have a minimum glycerine content between 4% and 8% and must comprise between 12% and 20% moisture content.

The preferred opaque soap bases are:

Tallow/Coconut Oil

Tallow Fatty Acids/Coconut Fatty Acids

Palm Oil/Palm Kernel Oil and fatty acids

Palm Oil Fatty Acids/Palm Kernel Fatty Acids/Palm Stearine

The composition of the opaque soap base used to produce translucent soap bars according to the invention may vary in terms of percentages of the components. For instance, the proportion of tallow and coconut oil can vary from 90:10 to 50:50, with percentages of 80:20 preferred, so long as the opaque soap base has a minimum glycerine content between 4% and 8% and a moisture content between 12% and 20%, so that translucency of the resulting bar soap is not adversely affected.

In accordance with a preferred embodiment of the present invention, a translucent soap bar comprises between about 92% to 98% of regular, opaque soap base and between about 2% to 8% of a selected benzoate ester or combination of benzoate esters. Preferably, the translucent soap bar comprises 96% opaque soap base and 4% of a selected benzoate ester. Typically, the total proportion of benzoate ester(s) added to the soap base will not exceed 6%, preferably being no more than 4% and not less than about 2% of the translucent soap bar. The invention also includes processes for making the translucent soap bars, in which the soap base and the benzoate esters are mixed together, i.e., admixed, at room temperature, and the mixture is adjusted to a moisture content in the range of 12% to 20%, following which the admixture may be processed, extruded, cut, and pressed to finished translucent soap bar form, using conventional soap finishing equipment.

The term “translucency” as used herein in reference to soap bars is typically determined by noting the point size of Roman type letters that can be read clearly through a parallel-faced slice of soap 3 millimeters thick. As set forth by F. V. Wells, Transparent Soap, Soap and Chemical Specialties, June 1955, translucency is measured by the ability to read bold-face type, of about 14 point size, through a quarter-inch thick cake of soap. A bar having a reading of 5 and above is considered to be translucent, whereas a bar having a reading of less than 5 is considered to be non-translucent, i.e., opaque. Table A below sets forth the results of translucency measurements of some commercially available soap bars, as follows:

TABLE A

| | TRANSLUCENCY |
|---|---|
| CAST SOAP | |
| Pears Soap, UK | 7.7 |
| Neutrogena Soap, dry skin formula, USA | 7.6 |
| Clavin Klein Soap, USA | 7.5 |
| EXTRUDED SOAP | |
| CD Soap (Original), Germany | 6.8 |
| Sensiciean Soap, France | 6.6 |
| Kappus Soap, Germany | 6.5 |
| San Francisco Soap, Co., USA | 6.5 |
| Bodyshop Soap, UK | 6.3 |
| Naturistics Soap, USA | 6.2 |
| Bodygraphy Soap, USA | 6.2 |
| CD Soap (New), Germany | 5.8 |
| Mild Transparent Seife, Germany | 5.6 |
| Boots Glycerine Soap, UK | 5.6 |
| Scottish Fine Soaps, UK | 5.3 |
| Pental Fruits Glycerin Bar, Australia | 5.2 |
| Komili Soap, Turkey | 3.7 |

Of the soap bars whose translucency is measured in Table A, the Pears and Neutrogena soaps are almost transparent as they are almost water clear. Thus, transparent soaps would have a value of 8.0 as determined by the Wells method of measuring translucency. Soaps with a value of 5 and above are considered translucent according to this assessment. All bars having values between 5 and 8 may be regarded both as translucent and transparent. Bars having a value below 5 are considered opaque.

The novel soap bars of this invention have unique properties in that they are substantially translucent, have good lathering and foaming characteristics, and cost substantially less to manufacture than translucent soap bars produced by known processes. The reduced cost of manufacturing compared to known methods of manufacturing translucent soap bars is a prime benefit of the process of the invention. The price of soap bars will decline as the moisture content of the bars increases.

The unique process for preparing translucent soap bars of the invention involving the use of benzoate esters provides for the delivery of other cosmetic benefits, such as emolliency, sun protection, and moisturizing properties, while ensuring clarity of the soap bar and superior afterfeel.

Benzoate esters having the following general structure

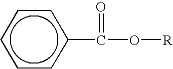

where R is a C12-C15 Alkyl, an alkyl group of C8 to C20, or a phenylethyl group

are suitable to improve translucency of opaque, translucent and transparent soap bases and soap bars, and as clarifying agents to clarify a clear base to improve translucency even more, and to clarify opaque or translucent soap bars or soap bases so as to significantly improve their translucency.

The preferred benzoate esters useful in making the translucent soap bars of this invention are added in amounts ranging from 2% to 8%, and preferably about 4%. The most preferred ester is FINSOLV® TPP (INCI name: C12-15 alkyl benzoate (and) dipropylene glycol dibenzoate (and) PPG-15 stearyl ether benzoate), followed by a combination of FINSOLV® TPP and FINSOLV® G-2 (INCI name: C16-17 alkyl benzoate).

The invention in its broadest aspect contemplates the use of specified benzoate esters as translucency-enhancing materials when they are mixed with regular, opaque soap base at room temperature, after which the mixture is adjusted for moisture content, and processed using conventional soap finishing equipment to form translucent soap bars.

The regular, opaque soap base useful as a starting material in the process of the invention is any opaque soap base produced by known opaque soap-making processes, i.e., fatty acid process, natural fat process, or semi-boil process.

Various opaque soap bases described herein may be used as the starting material in the process of the invention, but the invention is not limited to these soap bases. Preferably, the regular, opaque soap bases useful in making the translucent soap bars of this invention maybe obtained from various sources, such as Bradford Soap Works, Inc. of West Warwick, R.I. or The Dial Corporation of Montgomery, Ill., among other sources. Glycerine must be added to certain opaque soap bases before or during use in the process of the invention, if glycerine is extracted in preparing the base.

The translucent soap bars of the invention have superior physical and performance properties as compared to translucent soap bars produced by known processes, namely, translucency (Point Type Visible is between 5 and 10 on a scale of 1 to 10), superior foam characteristics, emolliency, improved soft skin feel, pleasant afterfeel, mildness, no grittiness, low cost, and biodegradable. It has also been found that the translucency of the soap bars of the invention does not deteriorate with storage, but actually improves.

A suitable regular opaque soap base plus a suitable benzoate ester, singly or in combination, can be made translucent using conventional soap finishing equipment. In manufacturing the translucent soap bars of this invention, a regular, opaque soap base is combined or admixed with a benzoate ester in a mixer called an amalgamator. Generally, the solid regular opaque or vegetable soap base is combined with the liquid benzoate ester in a dry blending process in amounts corresponding to from 92% soap base: 8% benzoate ester, and most preferably from 96% regular soap base: 4% benzoate ester, but can go as low as 98% soap base: 2% benzoate ester with excellent results. The admixing step is carried out in a continuous manner, in any conventional soap finishing line.

The process of “finishing” or producing translucent soaps is more sensitive than for opaque soaps. To achieve a satisfactory translucent soap bar, processing parameters set forth herein are adjusted for optimum processing for commercial production. The critical elements are:

Regular soap base must have between 12% to 20% moisture content, and preferably at least 16% moisture content;

Even and uniform mixing and refining of the regular, opaque soap base and benzoate ester is provided using an amalgamator/plodder/roll mill;

Roll mill/plodder cooling water temperatures are kept constant at between 15° C. and 35° C., and preferably between 25° C. and 30° C.;

Refining screens and/or drilled plates for each plodder are adjusted to optimize translucency. Preferably, screens having wire mesh of the following dimensions, or plates having openings of the following dimensions, are used:

| | |
|---|---|
| #1: | 0.15 mm |
| #2: | 0.03 mm |
| #3: | 0.05 mm |

Extrusion temperatures are optimized and maintained for each product formulation, in the range of 45° C. and 70° C.;

Recycle quantity of extra slugs, stamped bars are carefully controlled to minimize the amount of soap slugs recycled.

The process of the invention comprises the following steps:

Regular, opaque soap in the form of pellets or noodles are weighed off and placed in an amalgamator together with between 2% to 8% by weight of a specified benzoate ester or a combination of benzoate esters. The mixture is dry blended or admixed for between 2-6 minutes.

Following the amalgamation stage, the soap is refined, then compacted and extruded, and cut into slugs prior to stamping into soap bars in a desirable shape. By “refining” is meant the process of completing the mixing and making the soap more uniform, incorporating all ingredients into the base, using a refining plodder. During the compacting and extruding stages, the soap is compacted and a continuous slug or log of soap is formed, using an extruding plodder. By stamping is meant shaping the final bar. The process of the invention contemplates using conventional refining, plodding, and stamping processes and equipment.

Once the mixture is well combined in the form of a dry mix, the blend is transferred to the hopper of a roll mill or a Refiner/Plodder or a duplex refiner. The soap composition is refined using refining plodders fitted with screens preferably ranging from 0.15 mm to 0.05 mm mesh. Refining screens and/or drilled plates for each plodder may be modified to optimize translucency.

The parameters of the Refiner/Plodder are as follows:

| | |
|---|---|
| Cooling water temperature: | 20° C. to 40° C. |
| Extrusion head temperature: | 45° C. to 70° C. |

To produce translucent soap bars in large quantities, the soap composition is compacted using conventional soap finishing lines. The soap composition is processed through conventional soap finishing equipment, such as refiners and final vacuum plodders or continuously plodded into bars, which are then cut into small slugs that are stamped to obtain the final bars of soap. The extruded bars may be cut into slugs for pressing and pressed into finished translucent soap bars.

The translucent soap bars of the invention will typically contain between about 12% and 20% moisture, and preferably about 16% moisture.

The soap bars of the invention are translucent, have satisfactory lathering and foaming properties, are good cleansers and emollients, are hard, have an aesthetically pleasing appearance with good sheen or gloss, are stable against oxidation, decomposition, reaction with other soap ingredients, development of rancidity, and maintain their translucency during use. Their appearance and properties may be further improved by the addition of auxiliaries which are not detrimental to translucency and which are typically used in formulating soap bars. These include, but are not limited to, foam stabilizers, humectants, emollients, fragrances, anti-bacterial agents, dyes, pigments, pearlescent pigments, anti-oxidants, chelating agents, and vitamins. These additional materials may be added in their usual proportions and for their usual effect. The amount of these additives is usually 1% to 15% by weight in total, based on the total weight. These additives may be incorporated using conventional equipment at the time of mixing in the amalgamator.

No other agents are added or required to enhance translucency.

The translucent soap bars of the invention are advantageous in that they have the following properties: Water solubility/dispersibility; ease of emulsification; emolliency; lack of greasiness; pleasant skin feel; non-oily; superior foaming qualities; feel (wet and dry); lathering power; tactile properties; rinsing properties; cleaning characteristics; foam modifying properties; low surface tension; low sloughing and wear rate; bland odor; inert, essentially non-toxic and non-sensitizing; stability to light, heat, oxygen and hard water.

The invention may be employed to make translucent soaps, colored translucent soaps, pearlescent-translucent soaps, and combo bars.

The degree of translucency for the translucent soap bar of each example was determined using the Wells method described above. The translucency was measured at the values set forth in Table B, as follows:

TABLE B
TRANSLUCENCY OF SOAP BARS OF THE INVENTION

| | Soap Preparation | Translucency |
|---|---|---|
| Formulation I: | Control, i.e. opaque base | 0 |
| Formulation A: | Control + FINSOLV ® | 8 |
| | TPP (4% by weight) | |
| Formulation B: | Control + FINSOLV ® | 6 |
| | TN (4% by weight) | |
| Formulation C: | Control + FINSOLV ® | 6 |
| | BCO-115 (4% by weight) | |
| Formulation D: | Control + Glycerin | 8 |
| | FINSOLV ® TPP (4% by weight) | |
| Formulation E: | Control + FINSOLV ® | 7 |
| | TN (2% by weight) + FINSOLV ® | |
| | BCO-115 (2% by weight) | |

Any regular opaque soap base may be used, such as is set forth in Table C. Glycerin is added, if needed, as in Formulation D, to bring the glycerin content up to at least 8%.

Preferred opaque and translucent soap bases useful in the process of the invention are identified in Table C.

TABLE C
PREFERRED SOAP BASES

```
I. Source: Bradford Soap Works, Inc. (West Warwick, Rhode Island)
  A. Opaque soap bases:
      i. Tallow Traditional (85/15 - 80/20 - 70/30 -
        75/25 tallow:coconut oil)
      ii. Vegetable Traditional (80% palm oil; 20% coconut oil)
  B. Translucent soap bases:
      i. Tallow Translucent (85:15 tallow:coconut oil)
      ii. Vegetable Translucent (80:20 palm oil:coconut oil)
II. Source: The Dial Corporation (Montgomery, IL)
  A. Opaque soap bases:
      i. Opaque 85/15 Tallow/Vegetable Soap Base Pellets
      ii. Plastibar (NoMar) Opaque 85/15 Tallow/Vegetable
        Soap Base Pellets
      iii. Opaque 70/30 Tallow/Vegetable Soap Base Pellets
III. Source: Valley Products Co. (Memphis, TN)
  A. Opaque soap bases:
      i. Valpure ® 7525 PC Plus (vegetable soap base)
      ii. Valpure ® 8515 TC Plus (tallow soap base)
IV. Source: Marietta American Soap, Inc. (Olive Branch, MS)
  A. Opaque soap base:
      i. Tallow soap base (80% Tallow/20% Coconut Soap)
V. Hada Soap Corp. (Columbia, South America)
  A. Opaque soap base:
      i. Vegetable Soap Base (80/20)
VI. Cesar Iglesias (Santo Domingo, Dominican Republic)
  A. Opaque soap base:
      i. Tallow Soap Base (85/15)
```

Table C sets forth the preferred soap bases, but it is contemplated that other soap bases may be used in the process of the invention.

TABLE D
TRANSLUCENCY OF SOAP BARS OF THE INVENTION

| Ref. No. | Base/Type | Translucency¹ | Moisture % | Glycerin | Ester Added |
|---|---|---|---|---|---|
| 5040 | Transparent Base² | 9 | 18 | − | — |
| 5041 | Transparent Base | 9+ | 18 | − | Finsolv TPP |
| 5042 | Transparent Base | 10 | 18 | − | Finsolv TPP |
| 5062 | Taurus 134 85/15³ | 8+ | 18 | − | Surfine AZI-A; Finsolv SUN |
| 5074 | Taurus 134 85/15 | 8 | 17 | − | Finsolv TPP |
| 5123 | Veg. P-134 85/15⁴ | 8 | 18 | + | Finsolv TPP |
| 5130 | Taurus 134 85/15 | 7+ | 16 | − | Finsolv G2 |
| 5132 | Taurus 134 85/15 | 8 | 16 | − | Finsolv TPP; Finsolv G2 |
| 5133 | Taurus 134 85/15 | 8+ | 16 | + | Finsolv TPP; Finsolv G2 |
| 5136 | Taurus 134 85/15 | 8+ | 16 | + | Finsolv TPP; Finsolv G2 |
| 4949 | Taurus 134 85/15 | 8 | 17 | − | Finsolv TN; Finsolv BCO-115 |
| 4954 | Taurus 134 85/15 | 8 | 18 | − | Finsolv TPP |
| 4955 | Taurus 134 85/15 | 8 | 18 | − | Finsolv TPP |
| 5035 | Taurus 134 85/15 | 8 | 18 | − | Finsolv TPP |
| 5002 | Taurus 134 85/15 | 8 | 16 | − | Finsolv TPP; Surfine AZI-A |
| 5036 | Veg. P-134 85/15 | 8 | 17 | − | Finsolv TPP |
| 5037 | Veg. P-134 85/15 | 8+ | 17 | − | Finsolv TPP |
| 4990 | Reg. Base 85/15⁵ | 8+ | 18 | + | Finsolv TPP |
| 5050 | Transparent Veg. | 8+ | 16 | − | Finsolv TPP |
| | B⁶ | | | | |
| 5052 | Reg. Base 85/15 | 9 | 18 | + | Finsolv TPP |
| 4956 | Valley Reg. 85/15⁷ | 7 | 16 | − | Finsolv TPP |
| 5026 | Valley Reg. 85/15 | 8 | 16 | − | Finsolv TPP |
| 5058 | Valley Reg. 85/15 | 7+ | 16 | − | Finsolv TPP |
| 5054 | Regular opaque | 7 | 17 | + | Finsolv TPP |
| | base 80/20⁸ | | | | |
| 5056 | Regular opaque | 7 | 17 | + | Finsolv TPP |
| | base 80/20 | | | | |
| 5100 | Regular opaque | 7 | 19 | + | Finsolv TPP |
| | base⁹ | | | | |
| 5103 | Regular opaque | 7 | 18 | + | Finsolv TPP |
| | base | | | | |
| 5101 | Hada Veg. 80/20¹⁰ | 7 | 16 | + | Finsolv TPP |
| 5102 | Hada Veg. 80/20 | 7+ | 17 | + | Finsolv TPP |
| 5110 | Hada Veg. 80/20 | 7 | 17 | + | Finsolv TPP |

¹Translucency is measured on a scale of 1 to 10.
²Tallow Glycerin Transparent soap base from Bradford Soap Works, Inc. (West Warwick, Rhode Island)
³Tallow Traditional opaque soap base from Bradford Soap Works, Inc. (West Warwick, Rhode Island)
⁴Polaris 134 Vegetable Soap Base from Bradford Soap Works, Inc. (West Warwick, Rhode Island)
⁵Soap Base Pellets 85/15 tallow/vegetable opaque pellets from The Dial Corporation (Montgomery, IL)
⁶Transparent vegetable base from The Dial Corporation (Montgomery, IL)
⁷Regular tallow base from Valley Products Co. (Memphis, TN)
⁸80/20 tallow coconut opaque soap from Marietta American Inc.
⁹Regular Opaque soap base 85/15 from Cesar Iglesias (Dominican Republic).
¹⁰Hada Vegetable Soap Base 80/20 from Hada Soap Co. (Columbia, South America)

As can be seen in Table D, addition of benzoate esters causes opaque soap bases to become translucent and helps already translucent soap bases become even more translucent. In Table D, the amount of benzoate ester added is 4% by weight if only one ester was added; and 2% by weight each if a combination of two benzoate esters was added.

The process of the invention may be used to produce translucent soap compositions, both liquid and solid.

The process of making bar soaps comprises three main stages: (a) neutralization or saponification to produce neat soap (b) drying step to evaporate water from the neat soap, and (c) finishing lines to produce the bar soap.

As disclosed above, the benzoate esters of the invention may be added to the finishing line, i.e., to the opaque pellets in the mixer, and from there it is processed further through any of the duplex refiner, duplex vacuum plodder, TB cutter, press, wrapper, etc., to obtain a clear bar.

In a further embodiment of the invention, a translucency-enhancing amount of specified benzoate esters or combinations of two or more of these is incorporated in the saponification process to produce a soap base. The benzoate esters of the invention may be added earlier in the soapmaking process so as to make a translucent soap base even more clear or translucent. This comprises adding the benzoate esters of the invention to a translucent base either during saponification of neutral fats or during neutralization of fatty acids.

In a third embodiment of the invention, a translucency-enhancing amount of specified benzoate esters may be used as clarifying agents to clarify or render translucent already translucent soap base, so as to markedly improve its translucency. Clarification is the process of improving translucency by adding the benzoate esters of the invention to a clear soap base to produce an even more translucent soap. The benzoate esters maybe added at the neat soap stage, during saponification or neutralization, to improve clarity in the finished clear bar soap.

Saponification of neutral fats saponifies the triglyceride directly by boiling, by semiboiling, or by continuous processes. It is the process of making neat soap from opaque base, to obtain soap noodles or pellets. Neutralization of fatty acids is the method by which fatty acids obtained from natural triglycerides by hydrolysis and distillation are neutralized to make soap. Saponification is defined as the chemical reaction between fats, oils and caustic soda to form soap (neat soap) and glycerin. It is also the alkaline hydrolysis of fats to yield glycerol and soap. Neutralization of Fatty Acids is defined as the chemical reaction between fatty acids and caustic soda to form neat soap.

There are several continuous soapmaking processes, with substantially similar designs, which are commercially in use. The main differences between the soapmaking processes occur in the saponification stage. Commercial soapmakers use one or the other process, or a combination of elements of these, sometimes in combination with minor variations, which dictate the choice of saponification reactor, processing equipment and operational steps selected.

The benzoate ester or esters of the invention may be added at a point in the saponification or neutralization process calculated to produce optimum translucency in the resulting soap, which will vary depending on the type of soap to be produced, the raw materials and type of equipment used in the process. Preferably, the benzoate esters will be added at either one of two points in the saponification or neutralization process:

a. The benzoate esters may be added to the raw materials used to make the soap. That is, the benzoate esters may be added to the saponification or the neutralization steps. The ingredients are then run through the process to make neat soap.

b. Alternatively, the benzoate esters of the invention may be added at the point in the saponification or neutralization process where neat soap, a heavy paste containing 30%-35% water, is obtained. The neat soap including the benzoate esters are then heated in a dryer to 280° F.-300° F. to evaporate the water in the neat soap, to obtain soap pellets. If, in the course of a specific soapmaking process, it is found that the benzoate esters evaporate in the heated chamber, then the benzoate esters should be added at a later stage of the process, i.e., after the drying step.

Thus, the decision when to add benzoate esters to the soapmaking process is a matter of design choice. The benzoate esters may be added at different steps in the process to suit the particular process and equipment used, to optimize translucency.

It is understood that the embodiments described herein are merely exemplary and that a person skilled in the art may make many variations and modifications without departing from the spirit and scope of the invention. All such modifications and variations are intended to be included within the scope of the invention described herein.

What is claimed is:

1. A translucent soap bar comprising soap base and a translucency-enhancing amount of a benzoate ester.

2. The translucent soap bar of claim 1 wherein said benzoate ester has the general structure: 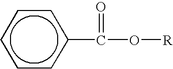
where R is a C12-C15 Alkyl, an alkyl group of C8 to C20, or a phenylethyl group.

3. The translucent soap bar of claim 2 wherein said benzoate ester is selected from the group consisting of:
(a) C12-C 15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.

4. The translucent bar of claim 1 wherein said translucency-enhancing amount of benzoate ester comprises from 2% to 8% by weight.

5. The translucent bar of claim 1 wherein said soap base is opaque.

6. The translucent bar of claim 1 wherein said soap base has a minimum glycerine content between 4% and 8% and comprises between 12% and 20% moisture content.

7. The bar of claim 1 wherein said soap base is selected from the group consisting of Tallow/Coconut; Tallow Fatty Acids/Coconut Fatty Acids; Palm Oil/Palm Kernel Oil and fatty acids; Palm Oil Fatty Acids/Palm Kernel Fatty Acids/Palm Stearine, and combinations thereof.

8. The bar of claim 7 wherein said soap base comprises a proportion of tallow and coconut oil in the ratio of 90:10 to 50:50.

9. The translucent soap bar of claim 1 further comprising nonoxynol-10 carboxylic acid.

10. A translucent soap bar comprising:
(a) a soap base having a minimum glycerine content between 4% and 8% and a moisture content between about 12% and 20%; and
(b) a translucency-enhancing amount of benzoate ester.

11. The translucent soap bar of claim 10 wherein said benzoate ester is selected from the group consisting of
(a) C12-C15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.

12. The translucent bar of claim 10 wherein said soap base is selected from the group consisting of Tallow/Coconut: Tallow Fatty Acids/Coconut Fatty Acids; Palm Oil/Palm Kernel Oil and fatty acids; Palm Oil Fatty Acids/Palm Kernel Fatty Acids/Palm Stearine, and combinations thereof.

13. A translucent soap bar comprising:
a. about 96% of an 80:20 tallow:coconut oil soap base having a minimum glycerine content between 4% and 8% and a moisture content between 12% and 20%, and
b. about 4% of a benzoate ester comprising C12-C15 Alkyl benzoate (and) dipropylene glycol dibenzoate (and) PPG-15 stearyl ether benzoate.

14. A translucent soap bar comprising:
a. about 96% of an 80:20 tallow:coconut oil soap base having a minimum glycerine content between 4% and 8% and a moisture content between 12% and 20%;
b. about 2% of a benzoate ester comprising C12-C15 Alkyl benzoate (and) dipropylene glycol dibenzoate (and) PPG-15 stearyl ether benzoate; and
c. about 2% of a benzoate ester comprising C16-17 alkyl benzoate.

15. A method of enhancing the translucency of a soap base comprising incorporating in said base a translucency-enhancing amount of benzoate ester.

16. The method of claim 15 wherein said benzoate ester is selected from the group consisting of
(a) C12-C15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.

17. The method of claim 15 further comprising incorporating in said base a translucency-enhancing amount of nonoxynol-10 carboxylic acid.

18. A continuous process for producing translucent soap bars comprising:
(i) between about 92% to about 98% by weight of soap base;
(ii) a translucency-enhancing amount of benzoate ester, about 2% to about 8% by weight;
said process comprising:
(a) admixing components (i) and (ii) in an amalgamator;
(b) refining the soap base by passing the soap base through a refining plodder provided with a fine screen;
(c) compacting and extruding the soap in a continuous log;
(d) cutting the soap into slugs; and
(e) stamping the slugs into finished translucent soap bar form.

19. The process of claim 18 wherein the moisture content of said soap base is adjusted to be in the range of 12% to 20% moisture.

20. The process of claim 18 wherein said benzoate ester is selected from the group consisting of
(a) C12-C15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.

21. A process for producing translucent soap bars comprising soap base, wherein the soap base is at least one of saponified and neutralized, and the resulting neat soap is dried, amalgamated, refined and formed in bars, the improvement comprising:
adding a translucency-enhancing amount of benzoate ester to said soap base.

22. The process of claim 21 wherein said soap base comprises a blend of tallow fat and coconut oil, or other fats and oils, or the corresponding fatty acids derived therefrom, useful in soap production.

23. The process of claim 21 wherein said soap base comprises a blend of from about 50:50 tallow:coconut fatty acids to from about 90:10 tallow:coconut fatty acids.

24. The process of claim 21 wherein said benzoate ester is selected from the group consisting of
(a) C12-C15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.

25. A translucent soap composition comprising
i) soap base;
ii) a translucency-enhancing amount of benzoate ester.

26. The translucent soap composition of claim 25 further comprising from about 1% to about 15% by weight of customary auxiliaries and additives.

27. The translucent soap composition of claim 25 wherein the customary auxiliaries and additives are selected from the group consisting of fragrances, dyestuffs, pigments, pearl pigments, bactericides, antioxidants, foam stabilizers, humectants, emollients, chelating agents and vitamins.

28. The translucent soap composition of claim 25 comprising
a) from about 92% to about 98% by weight soap base; and
b) from about 2% to about 8% by weight of benzoate ester.

29. The translucent soap composition of claim 25 further comprising glycerine in an amount, at a minimum, between 4% and 8% by weight, based on the weight of the composition.

30. The translucent soap composition of claim 25 wherein said benzoate ester is selected from the group consisting of
(a) C12-C15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.

31. The translucent soap composition of claim 25 wherein the soap composition has a moisture content between 12% and 20% by weight, based on the weight of the composition.

32. A method of improving the translucency of opaque, translucent and transparent soap base comprising adding a translucency-enhancing amount of benzoate ester to the soap base.

33. The method of claim 32 wherein said benzoate ester is selected from the group consisting of
(a) C12-C15 Alkyl benzoate (and) dipropylene glycol dibenzoate (and) PPG-15 stearyl ether benzoate;
(b) C12-C15 Alkyl benzoate;
(c) castor oil benzoate;
(d) C16-17 alkyl benzoate;
(e) Octyl Dodecyl benzoate;
(f) phenylethyl benzoate; and
(g) combinations thereof.
